(19) United States
(12) Patent Application Publication
(10) Pub. No.: US 2020/0229718 A1
(43) Pub. Date: Jul. 23, 2020

(54) NON-INVASIVE, REAL-TIME, BEAT-TO-BEAT, AMBULATORY BLOOD PRESSURE MONITORING

(71) Applicant: DP Holding (U.K) Limited, Grantham (GB)

(72) Inventors: Darren SPENCER, Grantham (GB); Peter BALMFORTH, Grantham (GB)

(21) Appl. No.: 16/651,749

(22) PCT Filed: Oct. 11, 2018
(86) PCT No.: PCT/GB2018/052909

§ 371 Date: Mar. 27, 2020

(30) Foreign Application Priority Data
Oct. 11, 2017 (GB) 1716661.2

(51) Int. Cl. A61B 5/021; A61B 5/024; A61B 5/0472; A61B 8/04; A61B 8/02; A61B 5/00; A61B 5/0215; A61B 8/00

(52) U.S. Cl. CPC A61B 5/02125; A61B 5/02438; A61B 5/0472; A61B 8/04; A61B 8/02; A61B 5/01; A61B 5/0022; A61B 5/02158; A61B 8/4236; A61B 5/4266; A61B 5/6833

(57) ABSTRACT

There is provided an ambulatory system, comprising at least first and second wearable sensors, for determining pulse transit time (PTT) between at least a first and at least a second fixed location within the cardiovascular system of a subject. The system comprises at least a first device, wherein the first device can contact the skin of the subject, the first device being positioned proximate to the first fixed location; and also comprises at least a second device, wherein the second device can contact the skin of the subject, the second device being positioned proximate to the second fixed location. The system further comprises a data collection module that is in communication with the first and second devices. The first device is configured to detect a timing cue within the cardiac cycle of the subject, and the second device is configured to detect a pulse pressure wave passing through the second fixed location. The data collection module collects data relating to the transition of the pulse pressure wave passing through the second fixed location, thereby enabling determination of a pulse transit time (PTT) between the first and second fixed locations.

### FIELD

The present invention is in the field of real time wearable sensor technologies that are used to monitor blood pressure (BP) including central blood pressure. Sensors may include electrocardiograms, other sweat analysis and/or body movement sensors. Live data feeds from such real time sensors can either be downloaded and read post recording or can deliver live data feed using Wi-Fi/4G/Bluetooth mobile telecommunications networks to remote devices.

### BACKGROUND

Non-invasive blood pressure monitoring in all its forms today typically relies upon decades-old sphygmomanometer measurement. According to this approach, an inflatable cuff applied to a limb or extremity is used create a supra-systolic pressure allowing measurement of systolic and diastolic pressure in the limb as the air in the cuff is released. In the doctors' surgery, or with home monitoring, this captures a single moment in time the blood pressure (BP) of the individual in a resting state. However, this measurement does not represent any variability in blood pressure that occurs through the day or night. 24-hour ambulatory BP monitoring can be used to gain a wider snapshot of BP variation throughout the day's activities. Nevertheless, this presents a challenge during the evening and at night as the devices are typically uncomfortable to wear, with the repeated cuff inflation/deflation cycles often waking the subject creating a “false representation” of night time and overall 24-hour blood pressure.

In addition, peripheral BP measurements have been shown to have variability between the limb (typically an arm) on which the device is placed, as the result of any vasculature differences between the two arms. This variability means that peripheral BP is just a reflective pressure of the central BP. Central BP, in contrast, can be used as a predictive value, and is far more representative of the extent of organ damage due to the effects of elevated BP.

It would be desirable to eliminate these challenges by collecting real-time, heartbeat-to-heartbeat central BP via one or more small, portable, and subject-friendly wearable devices.

During contraction of the heart, a longitudinal pressure wave is created that propagates outwardly along the vessel walls of the vasculature. The velocity of this longitudinal pressure wave, the pulse wave velocity (PWV), can be related to the elasticity of the arterial vessel walls and to their dimensions by the Moens-Korteweg equation. The Moens-Korteweg equation states that PWV is proportional to the square root of the incremental elastic modulus of the vessel wall given constant ratio of wall thickness, to vessel radius and blood density, assuming that the artery wall is isotropic and experiences isovolumetric change with pulse pressure. Hence, the PWV depends both on the arterial pressure and the intrinsic elastic properties of the arterial wall. The PWV can be determined from pulse transit time (PTT) which refers to the time taken by the pressure wave to travel between two arterial sites in the body of a subject. Importantly, the PWV has been found to be directly proportional to blood pressure in many circumstances. This is believed to be because an acute rise in blood pressure causes vascular tone to increase and hence the arterial wall to become stiffer causing the PTT to shorten. Conversely, when blood pressure falls, vascular tone decreases and PTT increases. (Smith et al. Thorax 1999;54:452-457). PWV can act as a biomarker for the measure of arterial stiffness and has direct correlations to morbidity and mortality. (Snellen “E. J. Marey and Cardiology: Physiologist and Pioneer of Technology (1830-1904)” Kooyker Scientific Publications; 1980, Laurent et al “Expert consensus document on arterial stiffness: methodological issues and clinical applications” Eur Heart J. 2006;27:2588-605) Changes in PWV have been directly linked to the development of increased arterial stiffness and vascular aging (Weber T et al. “Arterial stiffness, wave reflections, and the risk of coronary artery disease” Circulation. 2004;109(2):184-189).

PTT may be measured by recording the time interval between the passage of the arterial pulse wave at two consecutive sites. More recently, for ease of measurement, the electrocardiographic R or Q wave has been used as the starting point, as it corresponds approximately to the opening of the aortic valve. An estimation of the arrival of the pulse wave at a peripheral site, such as the finger, can be made using photoplethysmography (PPG). The PTT is defined as the time delay between the R-wave of the ECG and the arrival of the pulse wave in the periphery (finger). The R-wave is typically detected from a chest lead of an ECG, using amplitude and slope criteria. The arrival of the pulse wave is defined by the peak value of the differentiated signal, which corresponds to the steepest part of the ascent of the PPG signal in the finger.

According to this method, the PWV (cm/ms)=0.5×height (cm)/PTT (ms) when the middle finger is used as the peripheral site for PPG.

Conventionally the point on the photoplethysmograph pulse wave form which is either 25% or 50% (depending on which equipment is used) of the height of the maximum value is taken to indicate the arrival of the pulse wave. Using ECG leads and finger photoplethysmography, reproducible PTT measurements have been made very simply.

Gesche et al. (Eur J Appl Physiol, 10 May 2011) used this method of determining PWV to identify a correlation to systolic BP. The algorithm that they generated from studies on 13 human subjects demonstrated a correlation coefficient of r=0.89 for repeated measurements. Whilst this correlation is significant and shows promise, clearly there is a need for improved accuracy and reliability of measurement.

Other peripheral sites where an arterial wave form can be detected may be chosen, such as the ear lobe, though they are often less convenient and require adjustment of the equation for determining PWV on a person-by-person basis. Hence, whilst the equipment needed to measure this physiological signal is commercially available and relatively inexpensive it is unsuitable for long term ambulatory measurements.

Using the electrocardiographic R wave as a starting point, although convenient as it is easily identifiable on an ECG, does introduce an inaccuracy because there is a time delay between the occurrence of the R wave and the opening of the aortic valve (the so-called isometric contraction time). The PTT “measured” using existing PPG-based techniques therefore includes this time interval in addition to the time taken for the pulse wave to travel from the aortic valve to the periphery (“true” PTT). Isometric contraction time is itself influenced by the variables that affect PTT such as blood pressure and ventricular stroke volume. It is known that much of the lengthening in “measured” PTT during increased inspiratory effort can be the result of a prolongation of isometric contraction time rather than “true” PTT.

Another troublesome problem with current PTT measurement is also that of artefact. This is almost always due to interference with the PPG signal at the finger, but can also occur when chest wall movement disturbs the ECG leads. Such artefacts can usually be screened out if the signal is reviewed manually but, if automatic scoring is employed, then spurious interpretation can occur.

Hence, the current approach to measuring systolic BP via PPG apparatus hinders true ambulatory measurements. Motion artefacts are often introduced that require manual review, which is cumbersome, time consuming and prone to error. In addition, increased respiratory effort can cause increased measured PTT which is also artefactual according to current approaches based upon PPG.

There exists a need to overcome the current aforementioned problems in the art.

### SUMMARY

Described herein is a wearable sensor-based technology, typically in the form of a patch that can adhere to the skin of the subject using a hydrocolloid or equivalent biocompatible adhesive. The patch comprises an ultrasound based sensor which accurately monitors vascular BP. The sensor may monitor BP in a vessel selected from: aortic arch; descending aorta; inferior vena cava; superior vena cava; brachial artery; femoral artery and carotid artery or any combination of these locations, beat-to-beat. In some cases, the sensor monitors BP by ultrasound detection of the PTT, and thus the PWV, in the vessel.

The described wearable sensor-based device may further provide measurement of at least one of: surface ECG; movement (accelerometer); perspiration (sweat sensor); and temperature.

A cloud software based platform is also described, where results can be downloaded and analysed. In one iteration, this may be automated using Bluetooth/4G/Wi-Fi networks. This can provide tools for post monitoring evaluation and analysis, but in combination with current technology, can also provide real time, beat to beat BP monitoring.

In a more specific case, the present disclosure provides a device in the form of at least one ultrasound transducer (transmitter/receiver), typically configured as an ultrasound patch array. The ultrasound transducer of this case may comprise a plurality of contoured patches, placed at key echo window locations on the body of the subject. Placement of the patch array allows evaluation of blood flow as well as wall motion of an adjacent blood vessel, such as the aorta. By using a mathematical algorithm (e.g. a transformation function), accurate calculations in real time of the central BP of the subject can be determined, based upon the echo measurements of PTT, amongst other parameters, by the patch system. A plurality of ultrasound patches may be connected, in order to be able to accurately determine real time pre-load and after-load volumes as well as central systolic BP, central diastolic BP, central pulse pressure, postural changes associated with blood flow volume, and large artery/vein constriction and dilatation associated with blood flow and changes in homeostasis.

Hence, in a further described scenario, there is provided a patch based system of sensors and a recorder that continuously records the BP of a subject.

Further discussed herein are methods for monitoring the central BP of a subject using the described devices.

In a first aspect, there is provided an ambulatory system, comprising at least first and second wearable sensors, for determining pulse transit time (PTT) between at least a first and at least a second fixed location within the cardiovascular system of a subject. The system comprises at least a first device, wherein the first device can contact the skin of the subject, the first device being positioned proximate to the first fixed location; and also comprises at least a second device, wherein the second device can contact the skin of the subject, the second device being positioned proximate to the second fixed location. The system further comprises a data collection module that is in communication with the first and second devices. The first device is configured to detect a timing cue within the cardiac cycle of the subject, and the second device is configured to detect a pulse pressure wave passing through the second fixed location. The data collection module collects data relating to the transition of the pulse pressure wave passing through the second fixed location, thereby enabling determination of a pulse transit time (PTT) between the first and second fixed locations.

In some embodiments, the system is configured to determine a pulse wave velocity (PWV) measurement from the PTT, and/or is configured to determine a blood pressure measurement. The first device may comprise at least one sensor of surface electrocardiogram (ECG), and the timing cue may be the time of at least a part of the QRS complex of the ECG.

In some embodiments, the first device can comprise an ultrasound transducer. The first device may be configured to detect a pulse pressure wave passing through the first fixed location. This pulse pressure wave may be the timing cue. The second device may, additionally or independently, comprise an ultrasound transducer. Any of the ultrasound transducers may comprise a piezoelectric ultrasound transducer and/or a phased array imaging ultrasound transducer.

In some embodiments, the data collection module transmits data to a remotely located controller. The data collection module may comprise a controller. The controller may be configured to determine PTT, PWV and/or BP measurements, and may be configured to communicate one or more of these measurements to a user of the system. The controller may carry out analysis of the pressure waveform of any one or more of any pulse pressure waves detected by the system.

Any of the described devices may be comprised within a patch. For example, both the first and second devices can be comprised within a patch. Alternatively, the first device is comprised within a first patch and the second device is comprised within a second patch. Part or all of any of the patches can be implanted subcutaneously. The patches can also be located on the surface of the body of the subject. The patches can comprise a biocompatible adhesive, suitably a hydrocolloid adhesive. Contoured patches conforming to the anatomy of the subject can be used.

Any of the devices used may comprise an integral power supply. The devices may further comprise sensors configured to measure one or more of galvanic skin response, temperature, heart rate, photoplethysmography, and motion. Any one or more of the components of the system or devices of the system may be configured to communicate, with wireless communication, internally or externally.

In some embodiments, the system can comprise further devices. For example, the system can comprise a third device, or a third and a fourth device. These devices can contact the skin of the subject, and are positioned proximate to a fixed location, for example a third and a fourth fixed location. Any of these devices may be comprised within a patch as described.

Any of the fixed locations may be part or all of body structures selected from one or more of: aortic arch, descending aorta, inferior vena cava, superior vena cava, brachial artery, femoral artery and carotid artery. In some embodiments, the first fixed location is comprised within the heart, optionally the aortic valve. In some embodiments, any of the devices are positioned in registry with an ultrasound echo window, which may be selected from one or more of: apical long axis, suprasternal, parasternal long axis left ventricle, parasternal short axis aortic Valve level, posterior at the height of the aortic arch, posterior immediately superior to the iliac bifurcation, carotid artery left, carotid artery right, subcostal four chamber short axis (IVC), Right supraclavicular (SVC), brachial artery left, brachial artery right, femoral artery left, and femoral artery right.

In another aspect, there is provided a non-invasive method for determining PTT between at least a first and a second fixed location within the cardiovascular system of a subject. The method comprises positioning a first wearable sensor-based device proximate to the first fixed location, wherein the first device contacts the skin of the subject; and positioning a second wearable sensor-based device proximate to the second fixed location, wherein the second device contacts the skin of the subject. The method further comprises detecting a timing cue within the cardiac cycle of the subject via the first device; detecting a pulse pressure wave passing through the second fixed location via the second device; collecting data relating to the transition of the pulse pressure wave passing through the second fixed location, and thereby determining of a pulse transit time (PTT) between the first and second fixed locations.

The method may further comprise determining a PWV measurement from the PTT, and/or determining a blood pressure measurement. A step of analysing a pressure waveform of one or more of the detected pulse pressure waves may be included.

The devices used in the method may be further defined by any of the features described in relation to the first aspect, as appropriate.

In yet a further aspect, there is provided an ambulatory apparatus for determining pulse transit time (PTT) between at least a first and a second fixed location within the cardiovascular system of a subject, the apparatus comprising at least first and second wearable sensor-based devices. The apparatus comprises at least a first device, wherein the first device comprises a patch that can adhere to the skin of the subject, and at least one sensor of surface ECG, the first patch being positioned proximate to the first fixed location which is the heart; and also comprises at least a second device, wherein the second device comprises a patch that can adhere to the skin of the subject, and an ultrasound transducer, the second patch being positioned proximate to the second fixed location. The apparatus further comprises a data collection module that is in communication with the first and second devices. The first device is configured to detect a timing cue from the ECG, and the second device is configured to detect a pulse pressure wave passing through the second fixed location. The data collection module collects data relating to the transition of the pulse pressure wave passing through the second fixed location, thereby enabling determination of a pulse transit time (PTT), between the first and second fixed locations.

This apparatus may further comprise a third device; wherein the third device comprises a patch that can adhere to the skin of the subject, and an ultrasound transducer, the third patch being positioned proximate to a third fixed location and configured to detect a pulse pressure wave passing through the third fixed location. The second fixed location may be the carotid artery, and the third fixed location may be the femoral artery.

The apparatus may further comprise a fourth device; wherein the fourth device comprises a patch that can adhere to the skin of the subject, and an ultrasound transducer, the fourth patch being positioned proximate to a fourth fixed location and configured to detect a pulse pressure wave passing through the fourth fixed location. The fourth fixed location may be the brachial artery.

In some embodiments, the data collection module collects data relating to the transition of pulse pressure waves passing through the fixed locations, thereby enabling determination of a pulse transit time (PTT), between any combination of the fixed locations.

In a still further aspect, there is provided an ambulatory apparatus for determining pulse transit time (PTT) between at least a first and a second fixed location within the cardiovascular system of a subject, the apparatus comprising at least first and second wearable sensor-based devices. The apparatus comprises at least a first device, wherein the first device comprises a patch that can adhere to the skin of the subject, and an ultrasound transducer, the first patch being positioned proximate to the carotid artery; and also comprises at least a second device, wherein the second device comprises a patch that can adhere to the skin of the subject, and an ultrasound transducer, the second patch being positioned proximate to the femoral artery. The apparatus further comprises a data collection module that is in communication with the first and second devices. The first device is configured to detect a pulse pressure wave passing through the carotid artery, and the second device is configured to detect a pulse pressure wave passing through the femoral artery. The data collection module collects data relating to the transition of the pulse pressure waves passing through the carotid and femoral arteries, thereby enabling determination of a pulse transit time (PTT) between the carotid and femoral arteries.

The apparatuses of the latter aspects may be be further defined by any of the features described in relation to the earlier aspects, as appropriate.

### DRAWINGS

The invention is further illustrated in the accompanying drawings.

FIG. 1 shows a schematic view of the underside (skin contacting side) of a patch for use in a system of sensors for continuously recording the blood pressure of a subject according to one or more embodiments of the present invention;

FIG. 2 shows a schematic view of the underside of another patch according to a further embodiment of the present invention;

FIG. 3 shows an expanded view of the features comprised within the embodiment shown in FIG. 2.

FIG. 4 shows a schematic of a system according to some embodiments of the invention, wherein one or more patches are positioned on the body of a subject.

FIG. 5 shows a schematic of a system according to some embodiments of the invention, wherein information gathered from a subject is recorded and can be uploaded to a cloud system.

FIG. 6 shows a conceptual system (schematic) showing the major stages in the gathering of data and calculation of output values, according to embodiments of the invention.

FIG. 7A shows the ultrasound measurement of pulse wave arrival in a subject, at the carotid, brachial and femoral arteries.

FIG. 7B shows a graph of the time between the aortic valve opening at time 0 and the arrival of the pulse wave in the carotid, brachial and femoral arteries.

FIG. 7C shows the difference in the PWV measurement as determined by the ultrasound/ECG method, and by the standard cuff measurement.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

Prior to setting forth the invention, a number of definitions are provided that will assist in the understanding of the invention.

As used in this description, the singular forms “a,” “an,” and “the” include plural referents unless the context clearly dictates otherwise. Thus, for example, the term “a sensor” is intended to mean a single sensor or more than one sensor or to an array of sensors. For the purposes of this specification, terms such as “forward,” “rearward,” “front,” “back,” “right,” “left,” “upwardly,” “downwardly,” and the like are words of convenience and are not to be construed as limiting terms. Additionally, any reference referred to as being “incorporated herein” is to be understood as being incorporated in its entirety.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term ‘ambulatory’ as used herein means that the devices and or systems described herein are in some cases designed to be used by ambulatory patients, that is, patients who are mobile, and able to walk or otherwise move around. This means that the devices are portable, and can be used outside the clinic, without the need for constant connection to bulky external power sources or other equipment.

The term ‘ultrasound transducer’ refers to a device which can produce/transmit and receive ultrasonic waves, and can be used in ultrasonic scanning applications by interpreting reflected signals from a target. The term is intended to be synonymous with the terms ‘ultrasound transceiver’, ‘ultrasound sensor’ and ‘ultrasound probe’. The parts of the transducer which act as the transmitter and receiver may be separate or combined. Various frequencies of ultrasound can be used, depending on the depth of penetration required. The choice of ultrasound settings used may therefore depend on the location monitored by the transducer. For example, a 15-35 MHz transducer can be used, however, at least for monitoring of the brachial, carotid, and/or femoral arteries using pulse wave Doppler scanning techniques, frequencies of at least 0.5 MHz, suitably at least 1 MHz can be used. An advantage of using lower frequencies includes a reduction in power usage, which can prolong the life of the device and reduce the need for bulky power supplies.

The term ‘ultrasound window’ as used herein refers to an area on the body surface which allows effective ultrasound imaging of the underlying to be achieved. If an ultrasound transducer is placed ‘in registry with’ (that is, positioned close to and possessing a line of view that corresponds with the respective ultrasound echo window), such an ultrasound window, this can allow scanning of particular body structures.

The term ‘ECG sensor’ as used herein refers to apparatus for measuring an electrocardiograph (ECG), or the electrical activity of the heart. ECG recording measures the electrical signal generated by the propagation of ionic action potential currents in the heart fibres. Devices for measuring ECG data in a clinical setting include 12-lead, 5-lead, and 3-lead ECG devices. Portable devices for ECG, sometimes known as ‘Holter monitors’, are also known, and allow recording over a longer time period than stationary recordings. Devices for measuring ECG have been incorporated into adhesive patches for wearable, ‘on body’ and non-invasive recording of ECG; such devices are known and referred to herein as so-called ‘ECG patches’.

The classic features of an ECG trace include characteristic ‘waves’, referred to by letters, which indicate particular electrical events taking place in the cardiac tissue. The P wave represents depolarisation of the atria, spreading from the sinoatrial (SA) node towards the atrioventricular (AV) node. This wave usually appears as a relatively slow positive wave. The QRS complex (sometimes referred to as the R-wave) represents depolarisation of the ventricles (which also corresponds to ventricular contraction), appearing as small negative deflections either side of a large positive signal. Finally, the slow positive T wave represents the repolarisation of the ventricles. The QRS complex has the largest amplitude due to the relative size of the ventricles, and due to this, the R-R interval, being the time between the R peaks of this complex, is often used to measure heart rate, which calculated by the inverse of the R-R interval. The interval between the P-waves of ECG traces is also sometimes used for the measurement of heart rate.

The term ‘pressure wave form’ or ‘pulse wave form’ as used herein refers to a measurement of pressure, or a surrogate for a pressure measurement, over time in a particular blood vessel. The blood pressure inside any given blood vessel varies over the course of the cardiac cycle, in particular in the aorta and arteries, due to their function in carrying pressurised blood from the heart. In general, an arterial pressure wave form will have a peak corresponding to the high pressure of systole (heart contraction) and a trough corresponding to the lower pressure of diastole (heart relaxation and refilling).

The term ‘pressure wave front’ or ‘pulse wave front’, as used herein, refers to the arrival of a pressure change driven by heart ventricular contraction at a monitored blood vessel. The timing of the arrival of this wave may be measured in a number of ways.

The term ‘timing cue’ or ‘zero time point’ refers to a time point during a cardiac cycle to which the times of other detected events, suitably the arrival of a pressure wavefront in a particular blood vessel, are compared. Typically this time point precedes the times of other detected events. The timing cue can be an event in the ECG trace, such as the Q wave, the R wave, or the QRS complex. The timing cue can be the time of an event located in the heart itself, such as atrial or ventricular contraction or relaxation, or the opening of the aortic valve. Such events may be detected in various suitable ways, such as ECG measurement, auscultation, seismocardiography or ultrasound recording of heart activity. The timing cue can also be the time of an event located external to the heart, such as the arrival of a pressure wavefront in a particular blood vessel, such as the carotid artery, as measured by ultrasound scanning.

The term “pulse transit time” or “PTT” refers herein to the time taken for the pressure wave of each heartbeat to travel between two locations, suitably locations that have pre-determined by the operator of the systems and apparatus described herein, for example from the heart to a particular monitored blood vessel, or between two arterial locations. These locations can be referred to as ‘fixed locations’, although the precise location that is monitored may be dependent on the placement of the devices of the invention. For example, where the carotid artery is monitored, the location used for the calculation of PTT will be the portion of this vessel which is most effectively monitored by a device of the invention which is placed on the subject proximate to this location. The fixed locations can be relatively distant from each other, or can be adjacent. In cases where the timing cue relates to an event located in the heart, such as ventricular contraction or aortic valve opening, the PTT is the time elapsing between the timing cue and the detection of the arrival of a wavefront in the monitored blood vessel. In cases where the timing cue is a different event located in the heart, or is taken to be the time of an event located external to the heart, such as the arrival of a pressure wave in a particular blood vessel, the elapsed time may not correspond to the pressure wave travelling from the heart, and it may be necessary to adjust the elapsed time accordingly. Hence, it will be appreciated that the term ‘fixed’ refers to the choice of the operator to pre-determine the anatomical location or point where the sensors are positioned on the subject.

The term “pulse wave velocity” or “PWV” refers to the velocity of the pressure wave generated by the contracting heart and a particular blood vessel. It can be calculated from dividing the distance travelled by the pressure wave between two locations by the associated PTT. As above, if the timing cue corresponds to an event located external to the heart, distance can be measured between the locations of the timing cue and the monitored blood vessel. In such cases, it may be necessary to adjust the measured elapsed time, the distance between the two locations, or both, to compensate. For example, if the measured elapsed time corresponds to the difference between the time of wavefront arrival in the carotid and the femoral artery, the real travelled distance of the pressure wave can be estimated by the tape measure distance from the carotid to the femoral artery, multiplied by 0.8, (see Huybrechts et al “Carotid to femoral pulse wave velocity: a comparison of real travelled aortic path lengths determined by MRI and superficial measurements” J Hypertens. 2011 August;29(8):1577-82, and Bortel et al, “Expert consensus document on the measurement of aortic stiffness in daily practice using carotid-femoral pulse wave velocity” J Hypertens. 2011 December;29(12):2491).

The term ‘arterial stiffness’ refers to the degree of elasticity found in an individual's arteries. Increasing arterial stiffness may occur as a result of aging and atherosclerosis, and is associated with risk of cardiovascular events. PWV increases with arterial stiffness, and due to this relationship PWV is frequently used to monitor an individual's arterial condition.

The term ‘power supply’ can refer to any suitable means of supplying power to one or more electrical or electronic components such as ultrasonic transducers, ECG sensors and data collection modules. Suitable power supplies may include for example, cells, batteries including lithium-ion batteries, and the like.

The term ‘data collection module’ as used herein refers to any suitable means for collating, processing and/or storing data collected by the sensors of the invention The data collection module (50) may comprise a processor and data storage means, such as a flash memory. The data collection module (50) communicates with and collects the data from the sensors comprised in the devices of the invention, for example the ECG sensor and ultrasound transducer.

The term ‘subject’ as used herein refers to a human or animal to which the invention is applied. Typically the subject may be a human where blood pressure monitoring over time is desired. Various of the embodiments of the invention as described herein may be useful for application to humans as subjects, but also could be of use when applied to animals. Veterinary uses could include the monitoring of livestock, pets and other domestic animals, racehorses, show animals, animal being used in pharmaceutical and similar trials, and so on. Clearly, this will require significant amendments to be made with regards to calculations of blood flow distance and so forth, which would vary depending on the target animal.

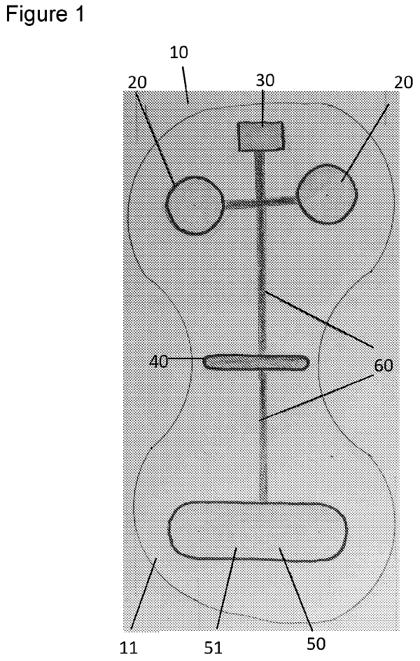

FIG. 1 shows a first embodiment of the invention, in which a device (10) comprises an adhesive patch (11) which allows the device to be applied to the skin of a subject. The patch comprises a number of components which are comprised within the area covered by the patch, thereby being placed in close or direct contact with the skin, in order to perform their functions. According to this embodiment of the invention, the components comprise at least one power cell (20), an ECG sensor (30), an ultrasound transducer (40) and a data collection module (50).

The adhesive patch (11) adheres to the skin of the subject using hydrocolloid or equivalent biocompatible adhesive. The adhesive patch (11) is preferably contoured and flexible, in order to conform to the shape of the subject. The patch (11) is configured to be attached in a particular orientation along the superior-inferior (or cranial-caudal) axis of the body, that is, with one end closer to the head, and the other closer to the feet. The adhesive patch may be applied at a single site on the upper torso of the subject, or additional patches may be applied at multiple sites on the subject's body to measure pressure wavefronts in different blood vessels. In embodiments where multiple patches are utilised, so-called patch array, the pressure wavefront may be monitored from a plurality of positions which allows improved correlation of determination of PWV. In one embodiment of the invention at least one device of the invention is applied proximate to the sternum, suitably proximate to the costal margin, xiphoid process and/or costal angle of the sternum of a human subject. In some instances, the patches are placed and configured to measure pressure waveforms in one or more of the carotid, brachial and femoral arteries.

The power cell (20) provides an integral power supply. The power cell (20) may be a lithium cell or battery and may be contained within a holder or other appropriate mounting assembly that is in electrical connection with the other components within the device.

The ECG sensor (30) is located at one end of the patch to be positioned in a superior/cranial location (towards the head, such that it is located superficial to the subject's heart. The ECG sensor (30) may be used to carry out measurement of the QRS complex of the ECG waveform to determine the onset of ventricular contraction and thus of the pulse wavefront.

The ultrasound transducer (40) is located centrally within the patch (11), in a more inferior/caudal location to the ECG sensor (30), such that it is located superficial to the descending aorta. Suitably, the ultrasound transducer (40) is a piezoelectrical transducer. In one embodiment the transducer may be a phased-array ultrasonic imaging transducer. The ultrasound transducer (40) is able to both send and receive an ultrasound signal and so detect the arrival of a pulse wavefront in the descending aorta (or other appropriate blood vessel), through a suitable ultrasound echo window. Hence, the device of the invention is capable of directly measuring the progression of the pulse wavefront through a major blood vessel within the subject's body. In one embodiment the device is able to determine the progress of the pulse wavefront directly by measuring the time taken for the pulse wavefront to progress across the field of the ultrasound echo window which incorporates the major vessel. In a second embodiment of the invention the measurement of the QRS complex of the ECG waveform may be used to determine the onset of ventricular contraction and, thus, initiation of the pulse wavefront, as a timing cue or ‘zero’ time point, with the arrival of the wavefront at the remotely positioned ultrasound echo window used to determine the end point. Hence, according this embodiment of the invention the PWV is calculated from the time elapsed between onset of ventricular contraction as determined by the QRS complex of the ECG waveform and the detection of the pulse wavefront in the ultrasound echo window, suitably at a location within the descending aorta, for example.

The data collection module (50) is located at the inferior end of the patch (11). The data collection module (50) may comprise a processor and data storage means, such as a flash memory. The data collection module (50) communicates with and collects the data from the ECG sensor (30) and ultrasound transducer (40). Communication between the data collection module (50) may occur via a wire, strip, ribbon or other suitable electrical connection. According to the device shown in FIG. 1, the electrical components (20, 30, 40, 50) are connected by an electrical strip (60), which preferably is flexible in order to maintain connections between the components despite changes in position or movement of the subject.

The data collection module (50) may simply act as a data store, as a wireless transmitter of data from the patch to a remote device, and/or may comprise a controller or processor that is capable of analysing data collected from the ECG sensor (30) and the ultrasound transducer (40). In the latter case the analysed data may also be stored within the data collection module or transmitted remotely. Analysis of the collected data may comprise calculating the PTT and the PWV, and thereby determining central BP in a real time, beat-by-beat basis. The data collection module (50) may further comprise a Wi-Fi, 4G, and/or Bluetooth network-enabled sender/receiver module (51) to compare data with devices located elsewhere, either on the subject or to transmit data to a cloud based software platform (not shown).

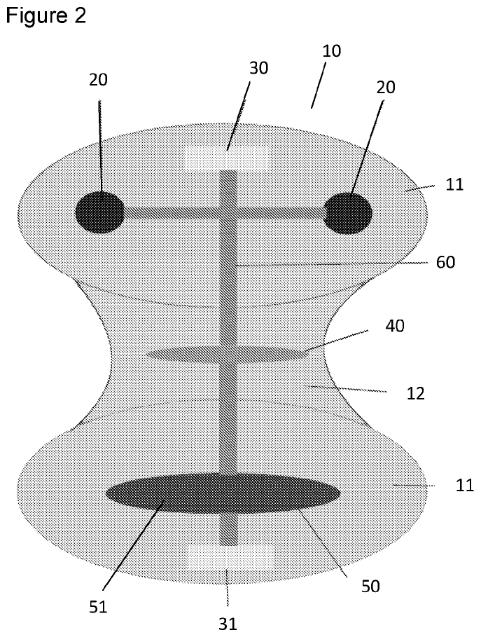

FIG. 2 shows a second embodiment of the invention, which comprises the features shown in FIG. 1, and comprises a first ECG sensor (30) located at the superior end of the patch (11) as well as a second ECG sensor (31) located at the inferior end of the patch (11). The second ECG sensor (31) works in combination with the first ECG sensor (30) to measure the surface ECG of the subject. In this embodiment the patch (11) comprises a central non-adhesive portion (12) within which the ultrasound transducer (40) is located. The adhesive patch (11) may be applied at a single site on the upper torso of the subject, or additional patches may be applied at multiple sites on the subject's body. Where multiple patches are utilised, in a similar manner to the earlier embodiment described in FIG. 1, the pressure wavefront may be monitored from a plurality of positions which allows improved correlation of determination of PWV. In such cases the PWV can be determined through comparison of wave arrival times in different blood vessels, for example, the carotid and femoral arteries. Under these conditions, the arrival of a particular wave in the carotid artery may constitute the ‘zero time point’ or timing cue. In a further embodiment, patches of the type described in FIGS. 1 and 2 may be used in combination within a patch array.

In other embodiments of the invention, a plurality of ECG sensors may be comprised within the device (10). According to such embodiments the patch may be oriented and positioned appropriately in order to optimise the collection of sensor data.

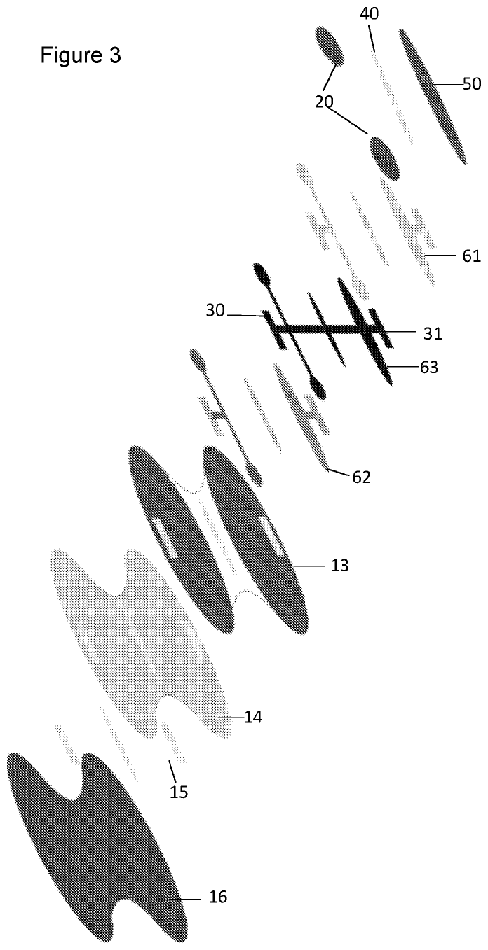

FIG. 3 shows an expanded view of the features comprised within the embodiment shown in FIG. 2. This shows that the patch (11) may be assembled from several layers including a structure/support material (13), an adhesive layer (14) using hydrocolloid or equivalent biocompatible adhesive, a hydrogel component (15) and an outer liner (16). FIG. 3 further shows that the electrical strip (60) which connects the components may further comprise two layers of electrical circuit insulator (61, 62) to create an electrical circuit (63).

In one embodiment, the invention incorporates a configuration wherein a plurality of patches (11) are applied to the subject, and work in combination through coordination of their data modules (50). The plurality of patches (11) may be interconnected via a cable system, or via Wi-Fi, 4G or Bluetooth sender/receivers (51) and cooperate to generate sensor data necessary to measure and accurately determine real time parameters such as those selected from: pre-load and after-load volumes; central systolic BP; central diastolic BP; central pulse pressure; postural changes associated with blood flow volume; and large artery/vein constriction and dilatation associated with blood flow and changes in homeostasis.

In some embodiments, the ultrasonic transducer (40) is positioned so as to monitor, via the appropriate ultrasound echo window, one or more blood vessels selected from: aortic arch; descending aorta; inferior vena cava; superior vena cava; carotid artery, brachial artery, and femoral artery, or any combination of these locations. In a further embodiment of the invention (not shown) the device (10) may operate in combination with a separate ambulatory ECG monitoring system, such as a conventional Holter device. Hence, in this embodiment the patch (11) may not need to comprise an integral ECG sensor (30) and may communicate with and receive ECG data directly from the ECG monitoring system.

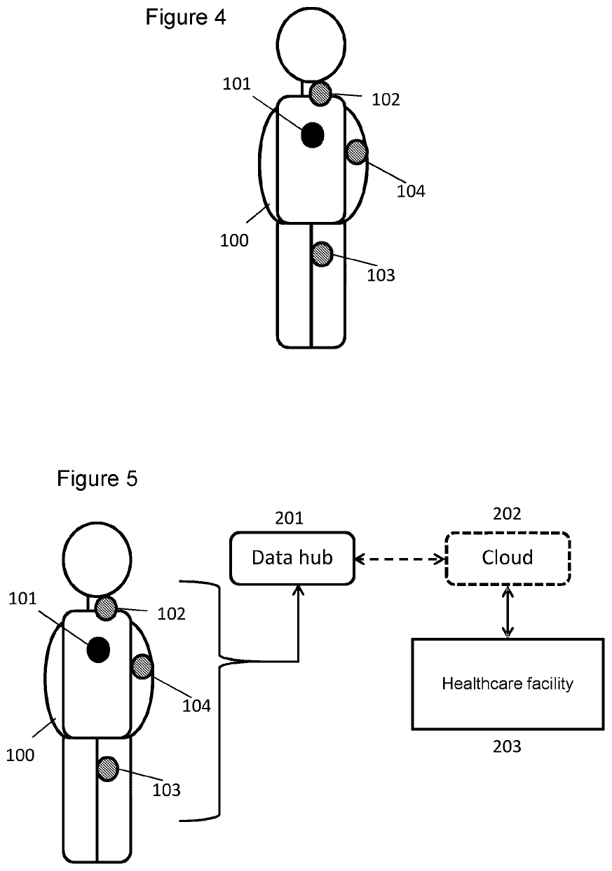

In one embodiment, there is provided a system comprising an ambulatory apparatus for applying to a subject, the apparatus comprising multiple patches which are applied to the subject on various parts of the body, and which remain in position for a period which may be of a duration of one or more hours, one or more days, or one or more weeks. The patches may have some or all of the features shown in FIG. 2, as appropriate. For example, in some embodiments of the described system (see FIG. 4) a patch comprising one or more ECG sensors (and with or without an ultrasound transducer) will be located over the heart (101) in order to record an ECG signal to serve as a timing cue, while one or more patches comprising ultrasound transducers but no ECG sensors are located over one or more arteries to be monitored, such as the carotid (102), femoral (103) and/or brachial arteries (104). In other systems there may be no patch comprising ECG sensors, but a plurality of patches comprising ultrasound transducers are be located over one or more arteries, and comparison will take place between the pressure measurements taken at these locations, with one selected as the location of the timing cue. In such systems the patches may comprise data modules which as above may communicate with each other to compare data and/or with a separate device so that information from multiple patches can be compared.

The apparatus acts to provide real-time monitoring of, for example, PTT, PWV and associated blood pressure estimates. These measurements may be made available to a user of the invention, such as the subject themselves, or a medical professional. As such, the apparatus may also comprise a display, which may be on an associated device for viewing by a user of the invention, or may transmit information via a wired or wireless system to a remote computer, to a remote or local storage device for later inspection, and/or to one or more so-called ‘smart’ device such as a telephone, laptop or tablet.

Such ambulatory apparatuses allow for blood pressure to be continually monitored under non-clinical conditions. This can allow instances of extreme blood pressure which might otherwise be asymptomatic to be detected, and the subject and/or a medical professional to be alerted. Similarly, blood pressure behaviour can be seen and/or recorded over long periods of time, allowing the detection of prolonged periods of abnormal levels, or trends of blood pressure readings over time.

This approach may be particularly useful when used to monitor the effect of particular treatments. Pharmaceutical and other treatments, for hypertensive or non-hypertensive conditions, may have effects on PWV and blood pressure, directly or indirectly, which may not be noticed at the time of a check-up in a clinical setting. As a result blood pressure can be viewed and/or recorded under various real-life conditions under particular circumstances, such as a change in a pharmaceutical strategy with a particular patient. This can allow outcomes like efficacy of hypertension treatments, or side effects on blood pressure of non-hypertension treatments to be measured, and can allow dosages to be revised in consequence.

A technical advantage is that the device of the invention is able to provide BP data in real-time via a minimal intervention approach to a medical sensing. This gives the subject the significant benefits of a comfortable, wearable device that does not inconvenience or interfere with their daily activities in order to gain a true representation of central BP.

According to yet further embodiments of the device of the invention, additional sensors may be comprised within the one or more patches (11), or in separate patches or devices, including, but not limited to: an accelerometer; pulse detecting sensors such as photoplethysmographs or pulse oximeters; galvanic skin response sensor (sweat sensor); sensors that measure sweat composition including glucose, lactate, sodium and potassium content in sweat; and thermocouple or thermistor (temperature). The additional sensor(s) may communicate with the data collection module (50) and provide supplementary physiological data that may be prognostic or diagnostic in value. For instance, changes in these data may correlate with particular blood pressure values (or vice versa), thereby allowing improved accuracy in the detection of any episodes of abnormal blood pressure.

The invention provides, in one or more additional embodiments, at least one non-invasive method for determining central, systolic and/or diastolic BP in a subject, comprising determining the PWV in a blood vessel located within the body of the subject via use of at least one ultrasound sensor applied to the skin of the subject. Suitably the ultrasound sensor comprises a piezoelectric ultrasound transducer, optionally a phased array imaging ultrasound transducer. In one embodiment of the invention the method is performed over a period of at least one hour, suitably at least two hours, at least six hours, at least 24 hours, at least 48 hours and not less than one week. In a further embodiment, the method is performed over a period of not less than one month, not less than six months, optionally for not less than one year.

In a specific exemplary embodiment of the invention, the entire system consists of two calibrated, standard automatic brachial blood pressure units that can measure right and left arm pressures simultaneously or separately via remote control. They are able to complete repeat readings and create BP averages and follow a pre-determined or programmable protocol to calibrate a combined sensor patch comprising a transmitter/receiver ultrasound array for the subject. The sensor patch may be connected to, or otherwise communicate with, a standard computer, or may be connected to a tablet-like or smartphone device for real-time monitoring and subject data input and calibration.

In one aspect, the device of the invention is a sensor patch that may comprise a contoured adhesive patch with an integral power supply (e.g. a lithium cell or battery) and appropriate ultrasound echo transducer for the location of the patch and the depth of field required. In one embodiment of the invention, the ultrasound transducer comprises a phased-array ultrasonic imaging transducer. The sensor patches may be connected to each other to facilitate communication of data and instructions, either via a cable system or via Bluetooth/Wi-Fi/4G and also to a recorder system. Each sensor patch may be specific to the location and contoured to fit that anatomy for the subject's comfort. The sensor patch is capable of monitoring, but not exclusive to and not limited to, all or any of the following standard ultrasound echo windows:
Apical long axis
Suprasternal
Parasternal long axis Left ventricle
Parasternal short axis Aortic Valve level
Posterior at the height of the aortic arch
Posterior immediately superior to the iliac bifurcation
Carotid artery Left
Carotid artery right
Subcostal four chamber short axis (IVC)
Right supraclavicular (SVC)
Brachial artery left
Brachial artery right
Femoral artery left
Femoral artery right

The ultrasound transducers comprised within the sensor patch monitor parameters such as: pulsatile blood flow, vessel wall motion, blood volume and so forth, to gather data necessary to determine a gated pulse wave from the left ventricle as the blood passes through the aortic tree. By using the QRS complex from a ventricular beat on a surface ECG as the timing cue, it is possible to accurately measure the time it takes for a single pulse of blood from the left ventricle to pass each of the one or more ultrasound patches. By combining the data from the respective ultrasound transducers, it is possible to directly measure large vessel wall motion; dilatation and constriction, blood flow and volume characteristics and be able to derive PWV, PTT, cardiac pre-load, cardiac afterload, central systolic BP, central diastolic BP, central pulse pressure, augmentation index, augmentation pressure, ejection time, heart rate, time to reflection, cardiac output, stroke volume and other cardiac indices.

Ultrasound methods of imaging blood vessels, and particularly methods of measuring blood flow in said vessels, may make use of the Doppler effect (Kisslo J A and Adams D B “Principles of Doppler Echocardiography and the Doppler Examination #1”. London: Ciba-Geigy. 1987). Ultrasound-interacting objects (such as components of the blood) can move relative to the ultrasound emitter, to approach or recede, thereby causing a positive or negative Doppler shift in the received echo. Changes in this measurement can indicate a change in flow rate within the imaged vessel. Measurements made in this way can be used to determine PWV with good agreement with other methods and can produce detailed readings of blood flow in monitored blood vessels over time (see for example Calabia et al. Cardiovascular Ultrasound 2011, 9:13).

In some embodiments of the invention an ultrasound transducer is located at the brachial/femoral artery, and in detecting by Doppler shift monitoring the change in blood flow caused by the heart, the onset of the pulse wave is determined. Methods to detect this can use continuous or pulsed ultrasound waves. While continuous waves can reliably measure relatively fast flow rates, they lack the ability to discriminate depth and therefore can be affected by noise from the whole tissue depth. Pulsed wave Doppler may therefore be of more use in the present context, since it can be tuned to detect data only from a certain depth.

Detection of Pulse Wave Arrival and Calculation of PWV

The pressure wave front caused by heart ventricular contraction can be determined in a number of ways, as is known in the field. The method used to determine an actual time point of pulse wave arrival for the calculation of PTT and PWV may depend on the quality of the data available. For a noisy trace it may be most reliable to use a thresholding measurement set above the level of background noise, with the time of the pulse wave arrival set by the trace exceeding the threshold. If cleaner and more detailed data is available, features of the waveform can also be measured, and in such cases details such as the peak can be used as a marker for the pulse wave arrival. Waveform analysis may be automatic, such as if carried out by a computer, or may require human input, such as a medical professional. In some cases automatic analysis can be moderated by input from a human user and/or improved automatic algorithms. Similar approaches can be used to determine other features such as the timing cue, where it is derived from an ECG trace. Many methods of automatically determining parts of an ECG trace are likewise found in the art, for example NEMon software.

Once the timing of the measured events is determined, the time elapsing between the timing cue and the measured event can be easily calculated by subtraction, to give the PTT. This value is then generally used in calculating the PWV. In some cases PWV can be determined directly, from tracking the progression of a pressure wave across a window monitored by an ultrasound sensor. Often, however, PWV is calculated from dividing the distance travelled by the blood along the vascular system by the PTT measurement taken for the pressure wave to travel that distance.

Measurement or estimation of distances travelled by blood in the vascular system is needed to determine PWV from a PTT measurement. While powerful methods such as magnetic resonance imaging (MRI) scans can be used to accurately measure the travel path of various blood vessels, this is not always practical to carry out for each subject. Other ways of estimating distances can be used, such as tape measurements on the body surfaces, or values based on the subject's height and/or weight. Various studies have been used to improve the calculations involved in these estimates to gain more accurate measurements of aortic travel distance, and so gain a more precise reading. In 2010 Nemeth et al (“The Method of Distance Measurement and Torso Length Influences the Relationship of Pulse Wave Velocity to Cardiovascular Mortality” Am J HTN, Vol. 24 Number 2, 155-161 February 2011) showed that by measuring the distance from the supra-sternal notch to the femoral artery and subtracting it from Supra sternal notch to carotid artery distance, a more precise measure of the distance between these structures could be obtained. More recently, Huybrechts et al demonstrated that the tape measure distance from the carotid to the femoral artery, multiplied by 0.8, corresponds best with the real travelled aortic path length (see also Bortel et al).

Calculation of Blood Pressure Measurement

There is a considerable body of work regarding the possible ways of determining blood pressure measurements from PWV and/or PTT values.

Some methods make use only of the PWV as an input, relying on the apparent proportional relationship between blood pressure and PWV, at least at relatively normal physiological ranges (the correlation reducing in accuracy at extremes). This relationship has been derived experimentally, and also theoretically, from the Moens-Kortweg and Bramwell-Hill equations, which relate arterial wall elasticity in terms of compliance and pressure, to PWV (Mukkamala et al. “Towards Ubiquitous Blood Pressure Monitoring via Pulse Transit Time: Theory and Practice”, IEEE Trans Biomed Eng. 2015 August;62(8):1879-901). Since PWV and PTT are inversely proportional to each other, blood pressure can also be said to be inversely proportional to PTT. The inverse relationship can be expressed as follows:

$$Blood pressure = \frac{{K}_{1}}{PTT} + {K}_{2}$$

where K₁ and K₂ are unknown, subject-specific values. While attempts at using non-linear relationships have been attempted, these involve multiple unknowns, which are difficult to determine for each subject. However, even with the relatively straightforward linear model, there are very significant differences between individuals which therefore importantly require important calibration steps for each subject under various conditions, allowing the constants in the equation to be determined.

In order to measure BP in each individual subject accurately via this non-invasive technique, the system may undergo a calibration step as part of the subject set up. The setup may comprise use of a standard digital brachial pressure cuff that provides standard measures of Systolic BP, diastolic BP, pulse pressure and heart rate. Once a standard calibration has been undertaken that communicates directly with the sensor patch, the mathematical transformation function may be applied to data acquired from the sensor. Recent clinical data has been able to accurately demonstrate that the calculation of Pulse Wave Velocity (PWV) alone, by using an appropriate mathematical transformation function, that enables the values for central systolic BP and central pulse pressure to also be determined. These values of measuring central BP have a more significant prognostic value of end organ damage than peripheral BP (Sueta et al, IJC heart and vasculature 8 (2015); 52-54).

Best practice for calibration may include the standard technique of blood pressure measurement, with the subject resting for 5 minutes on a seated stool in a quiet room with their non-dominant arm being measured 3 times sequentially, with the average of 3 recordings being used as a baseline. It may be prudent to measure the subject in several positions to gain a better accuracy for the various activities that can be measured if an accelerometer is comprised within the sensor patch of the invention. By way of example, baseline measurements may be taken whilst the subject is sitting, standing and supine, with 3 recordings being used at each position and the average (mean) of each being used. It may also be prudent to measure both left and right arm simultaneously in the various positions.

A pre-determined calibration program may be followed, as set out below:
1. discuss procedure with subject and gain any necessary consent
2. place on at least one sensor patch, whilst subject is lying down
3. test device connection to peripheral blood pressure recorder
4. have subject lying down for 5 minutes
5. automatic BP recording and upload of averages to sensor patch to capture data with a accelerometer/body position data
6. subject to stand for 1 minute
7. automatic BP recording and upload of averages to sensor patch to capture data with an accelerometer/body position data
8. subject to sit in a stool with arms relaxed on tables by their side for 1 minute
9. automatic BP recording and upload of averages to sensor patch to capture data with an accelerometer/body position data
10. disconnect patient from calibration unit.

The above represents one particular calibration protocol and is no way limiting upon the methods or apparatus of the invention. Calibration estimates are, of course, more accurate with more pairs of blood pressure and PWV/PTT measurements. Further methods of perturbing blood pressure which can be used include Cold pressor (immersing the subject's hand or limb in cold water), physical exercise, mental arithmetic, sustained handgrip, controlled breathing, and pharmaceutical interventions such as nitroglycerin. These can lead to greater perturbations of blood pressure than postural changes alone and so improve calibration.

Methods of measuring blood pressure from PWV can also involve relatively detailed analysis of the pressure waveform itself. This can allow more information to be obtained, but does require an accurate picture of the pressure waveform to be available.

For instance, this can allow detection of different PTT associated with different parts of the pressure waveform. Blood pressure varies over the cardiac cycle, for example from 80 mmHg (diastolic) to 120 mmHg (systolic). This means that the PTT will differ for different parts of the pressure trace. If a detailed report of the waveform is available, then multiple PTT/PWV values can be generated under the same conditions, with the PTT for the highest pressure corresponding to the systolic blood pressure, and the lowest to the diastolic pressure. This approach also requires that separate timing cues are obtained for diastole and systole for accurate comparisons to be made.

Algorithms which act to calculate blood pressure from pressure data gathered by ultrasound can be developed centrally and applied to the data generated by the invention. For example, patients undergoing cardiac catheterisation (specifically left heart cardiac catheterisation) may have fitted internal catheters which enable pulse wave arrival, PTT and PWV data, and central blood pressure to be measured directly, albeit in a clinical setting. Such patients could also have ultrasound data simultaneously gathered with devices or systems according to the present invention. The data generated by the catheters could then be used to determine the features of the concurrent ultrasound trace which relate to features such as the arrival of the pulse wave. Combining the internally measured, central measurements with the data gathered by the applied patches, will allow for a better baseline to which independently gathered patch data can be compared. This baseline can be continually updated as further data is collected. An example of this kind of system can be seen in FIG. 5, where data gathered from a healthcare facility (203), is uploaded to a cloud based service (202), and the developed algorithms used to determine features of ultrasound traces gathered by ambulatory systems according to the invention. Oversight can be maintained which allows for the disposal of spurious information.

Given that the invention can allow for the prolonged and continuous recording of hundreds of heartbeats, and associated PWV and blood pressure calculations, calibration can continue over time for each subject, so that the model used to calculate blood pressure can be updated. In addition, data from multiple subjects can be pooled so that the impact of other contributory factors can be taken into account, for example sex, ethnicity, body mass index (BMI), smoking status, and so on. These data can feed into a computer model developed over time with multiple subjects, in order to develop an enhanced, better calibrated model.

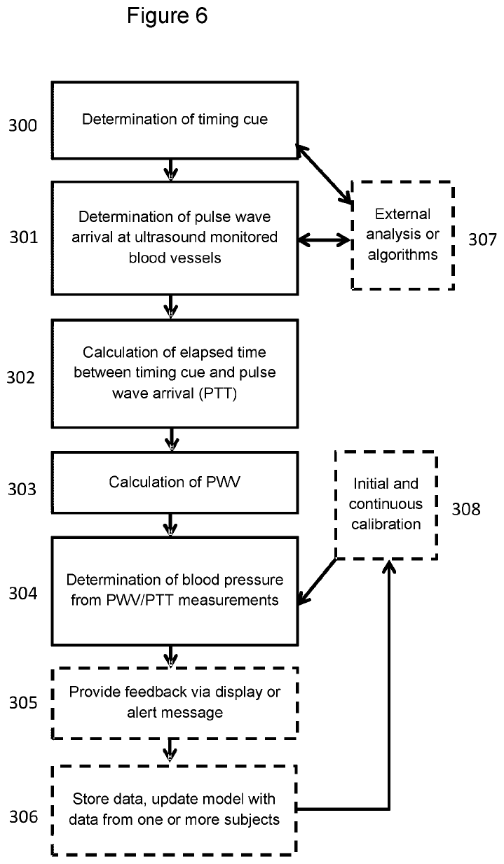

FIG. 6 shows a conceptual system summarising certain of the steps involved in the functioning of the invention in some embodiments. One or more of the steps may occur locally, for example in processors found within or separate to the data collection modules of the system of the invention, or remotely, for example in a remote server or cloud-based system. The timing cue is determined (300)—this can be for example part of an ECG trace measured by an ECG sensor, or a feature of a pressure wave in a monitored artery as measured by an ultrasound transducer forming part of the invention. Ultrasound data from one or more monitored blood vessels is obtained and analysed (301) to determine the timing of measured events such as pulse wave arrival and/or other features of the trace. As discussed, analysis of ultrasound data may occur with input from external sources (307), such as algorithms developed by healthcare facilities. PTT values are determined (302) from comparing the timing cue and the measured events, and PWV values are calculated (303), using stored distance values determined at calibration. A blood pressure measurement is determined, for example by one or more of the methods discussed above (304), which may be subject to calibration, either from an initial calibration stage or an ongoing updated model (308). This blood pressure measurement may be used to provide feedback to a user of the invention via any suitable means (305). The data generated by the process may be stored locally or remotely, and may be used to revise a model for the same subject, or to feed into a model to be used for multiple subjects (306).The aforementioned embodiments are not intended to be limiting with respect to the scope of any claims, which may be filed on applications filed in the future and claiming convention priority from this application. It is contemplated by the inventors that various substitutions, alterations, and modifications may be made to the invention without departing from the spirit and scope of the invention as defined by the claims.

### EXAMPLE

The following example was carried out to determine whether standard Doppler echocardiography can accurately capture blood flow in the carotid artery, brachial artery and femoral artery. Additionally, the example aimed to determine whether Pulse Wave Velocity be determined using the same technique in conjunction with a fixed stable reference point, namely the Q-wave of an electrocardiogram (ECG).

Methods:

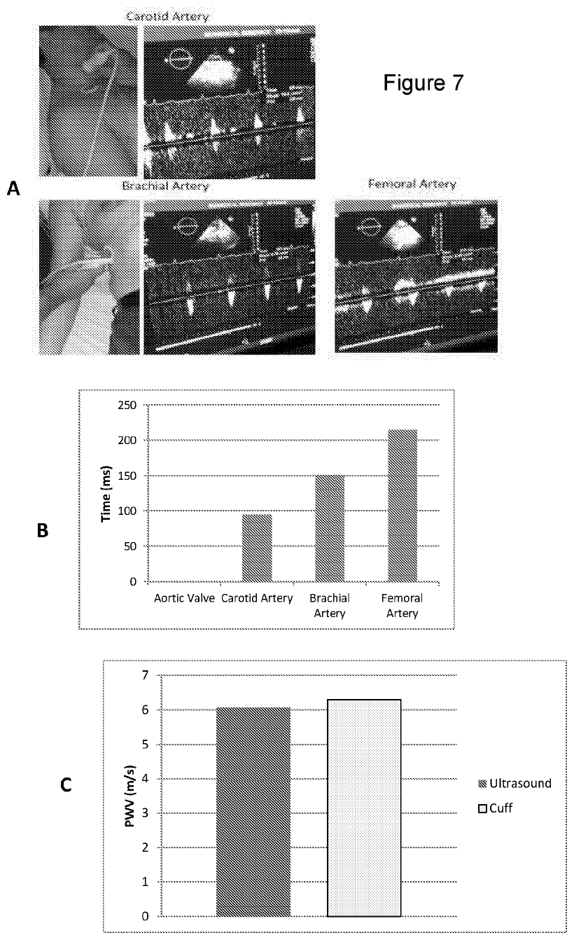

A 41 year old, healthy male subject underwent both pulse wave and continuous wave Doppler measurement over the carotid artery (CA), brachial artery (BA) and femoral artery (FA). The same 15-35 MHz probe (Philipps 1E33) was used to measure both pulse wave and continuous wave Doppler (FIG. 7A). A surface ECG was used to provide a stable reference for the onset of the blood pressure wave front. The Q-wave of the ECG was taken to be a surrogate for the opening of the aortic valve, as this is an easily-distinguished portion of the ECG trace which is locked to a particular part of the cardiac cycle.

Results:

Using continuous wave and pulsed wave Doppler in a stable patient, a timing interval was determined from a stable QRS reference to the blood pressure wavefront measured in the target artery, as measured by detection of a deviation from the baseline reading. No difference was seen between the two ultrasound wave modalities.

TABLE 1
The measured timing intervals from each of the locations
as measured from the stable QRS reference (see FIG. 4B)

| | Time (ms) | Velocity (cm/s) |
|---|---|---|
| Q wave to Carotid Artery | 95 | 15.6 |
| Q wave to Brachial Artery | 151 | 9.78 |
| Q wave to Femoral Artery | 215 | 2.3 |

The distance between the carotid and the femoral artery was measured, and multiplied by 0.8, in order to estimate the aortic path length (Huybrechts et al), giving a value of 73 cm. By adding the measured distance from the aortic valve to each of the arterial positions, a pulse wave velocity (PWV) can be determined by using the standard formula, where t is the transit time measured between the two points:

PWV=Distance traveled/t

At the time of recording, the blood pressure (as measured by a standard inflation cuff) was 128/85 mmHg. The online PWV calculator from the University of Ghent, Belgium was used to calculate the subject's PWV (http://www.biommeda.ugent.be/research/multiphysics-modeling-and-cardiovascular-imaging/calculator-assessment-measurements-carotid). This uses the time difference in waveform arrival between the carotid and the femoral arteries (in this case equal to 215−95=120 ms). A PWV of 6.08 m/s was calculated (see Table 2), given the measured distance of 73 cm.

TABLE 2
Subject data and PWV determination

| | |
|---|---|
| Age | 41 |
| Systolic BP (mmHg) | 128 |
| Diastolic BP (mmHg) | 85 |
| Carotid-femoral distance (cm) | 73 |
| Transit time (ms) | 120 |
| Mean Arterial Pressure (mmHg) | 102 |
| PWV (m/s) | 6.08 |

To verify the accuracy of the measurement, a calibrated device, certified by the European Society of Hypertension, was used to measure PWV. Using the Mobilograph (IEM, Bonn, Germany) 24 hour Ambulatory Blood Pressure Monitor in testing mode, a PWV from a cuff based system was measured immediately after the echocardiography recordings. Over a quiet 5 minute period, a PWV of 6.3 m/s was measured with this equipment.

Discussion:

The above example was conducted to replicate work demonstrating that Doppler ultrasound, in a stable, resting individual can measure approaching wave fronts.

The data clearly demonstrate a timing difference from the fixed ECG reference point to the CA, BA and FA. By adding the known, measured distance from the atrial valve to CA/BA/FA, a pulse wave velocity can be determined. The data were corroborated with that of a proven and tested system, achieving very similar PWV results (6.08 m/s vs 6.3 m/s, FIG. 4C). Possible sources of this difference may be accounted for by the different methods of data collection (echocardiographic vs cuff based), different mathematic algorithms used to derive the PWV, the patient position and posture during the recordings (lying vs sitting) or the accuracy of the measurement of distance from carotid to femoral artery.

Whilst in this investigation an ECG measurement was used as a timing reference, it may be feasible that the wavefront measured at the CA could be used as a time 0 and the time difference between this and the wavefront at the FA would then be the only measurement required in order to calculate PWV accurately.

Whilst no differences were seen between pulse wave and continuous wave Doppler in this example, the pulse wave Doppler has certain advantages for the present application, as the vessels are relatively fixed in location and not especially deep within the tissue, so this approach may give better clarity. In comparison the use of continuous wave Doppler is extremely effective at scanning wide and/or deep areas, but can be associated with increased artefact generation, due to its inability to distinguish and exclude different depths.

In conclusion, using standard echocardiographic techniques and equipment, combined with a stable surface ECG reference, a timing interval can be accurately measured to demonstrate the velocity of blood flow from the heart to the brachial, carotid and/or femoral arteries. Using standard mathematical calculations, these values can be easily converted to determine Pulse Wave Velocity.

1. An ambulatory system comprising at least first and second wearable sensors for determining pulse transit time (PTT) between at least a first and at least a second fixed location within the cardiovascular system of a subject, the system, comprising:
at least a first device, wherein the first device can contact the skin of the subject, the first device being positioned proximate to the first fixed location;
at least a second device, wherein the second device can contact the skin of the subject, the second device being positioned proximate to the second fixed location,
a data collection module that is in communication with the first and second devices;
wherein the first device is configured to detect a timing cue within the cardiac cycle of the subject, and wherein the second device is configured to detect a pulse pressure wave passing through the second fixed location, and wherein the data collection module collects data relating to the transition of the pulse pressure wave passing through the second fixed location, thereby enabling determination of a pulse transit time (PTT) between the first and second fixed locations.

2. The ambulatory system of claim 1, wherein the system is configured to determine a pulse wave velocity (PWV) measurement from the PTT.

3. The ambulatory system of claim 1, wherein the system is configured to determine a blood pressure measurement.

4. The ambulatory system of claim 1, wherein the first device comprises at least one sensor of surface electrocardiogram (ECG).

5. The ambulatory system of claim 4, wherein the timing cue is the time of at least a part of the QRS complex of the ECG.

6. The ambulatory system of claim 1, wherein the first device comprises an ultrasound transducer.

7. The ambulatory system of claim 6, wherein the first device is configured to detect a pulse pressure wave passing through the first fixed location.

8. The ambulatory system of claim 7, wherein the timing cue is a pulse pressure wave passing through the first fixed location.

9. The ambulatory system of claim 1, wherein the second device comprises an ultrasound transducer.

10-18. (canceled)

19. The ambulatory system of claim 1, wherein the first and second devices are comprised within a patch.

20. The ambulatory system of claim 1, wherein the first device is comprised within a first patch and the second device is comprised within a second patch.

21. The ambulatory system of claim 20, wherein a part or all of one or more of the patches are implanted subcutaneously.

22. The ambulatory system of claim 20, wherein one or more of the patches are located on the surface of the body of the subject.

23. The ambulatory system of claim 20, wherein one or more of the patches comprise a biocompatible adhesive.

24. The ambulatory system of claim 23, wherein the biocompatible adhesive is a hydrocolloid adhesive.

25-26. (canceled)

27. The ambulatory system of claim 1, wherein the first fixed location is comprised within the heart, optionally the aortic valve.

28. (canceled)

29. The ambulatory system of claim 1, wherein one or more of the devices also comprise sensors configured to measure one or more of galvanic skin response, temperature, heart rate, photoplethysmography, and motion.

30-31. (canceled)

32. The ambulatory system of claim 1, wherein any of the fixed locations are part or all of body structures selected from one or more of: aortic arch, descending aorta, inferior vena cava, superior vena cava, brachial artery, femoral artery and carotid artery.

33. system of claim 1, wherein any of the devices are positioned in registry with an ultrasound echo window.

34. The ambulatory system of claim 33, wherein the ultrasound echo window is selected from one or more of: apical long axis, suprasternal, parasternal long axis left ventricle, parasternal short axis aortic Valve level, posterior at the height of the aortic arch, posterior immediately superior to the iliac bifurcation, carotid artery left, carotid artery right, subcostal four chamber short axis (IVC), Right supraclavicular (SVC), brachial artery left, brachial artery right, femoral artery left, and femoral artery right.

35-45. (canceled)
